(19) United States
(12) Patent Application Publication
(10) Pub. No.: US 2023/0039829 A1
(43) Pub. Date: Feb. 9, 2023

(54) ASSESSING HEMODYNAMICS USING ELECTRICAL IMPEDANCE MEASUREMENTS

(71) Applicant: Timper Medical B.V., Eindhoven (NL)

(72) Inventor: Daniela Davi Madureira Iope, Sao Paulo (BR)

(21) Appl. No.: 17/758,057

(22) PCT Filed: Dec. 28, 2020
(86) PCT No.: PCT/IB2020/062492

§ 371 Date: Jun. 27, 2022

Related U.S. Application Data
(60) Provisional application No. 62/954,885, filed on Dec. 30, 2019.

(51) Int. Cl. A61B 5/0536; A61B 5/08

(52) U.S. Cl. CPC A61B 5/0536; A61B 5/0809

(57) ABSTRACT

Disclosed herein are systems, non-transitory computer readable media, and methods to employ electrical impedance-based devices in clinical setting to perform hemodynamic assessments. A system may include a plurality of electrodes; and a controller coupled to the plurality of electrodes. The controller may receive a sequence of impedance datasets. The controller may generate a corresponding impedance image. The controller may determine a pre-maneuver hemodynamic measurement from a first region of interest (ROI) from at least one impedance image prior to a maneuver. The controller may determine a post-maneuver hemodynamic measurement from the first ROI from at least one impedance image following the maneuver. The controller may receive at least one parameter associated with the maneuver. The controller may determine a hemodynamic figure of merit based at least on the pre-maneuver hemodynamic measurement, the post-maneuver hemodynamic measurement, and the at least one parameter associated with the maneuver.

### CROSS-REFERENCE TO RELATED APPLICATIONS

This application is a national phase entry under 35 U.S.C. § 371 of International Patent Application PCT/IB2020/062492, filed Dec. 28, 2020, designating the United States of America and published as International Patent Publication WO 2021/137136 A1 on Jul. 8, 2021, which claims the benefit under Article 8 of the Patent Cooperation Treaty to U.S. Patent Application Ser. No. 62/954,885, filed Dec. 30, 2019.

### TECHNICAL FIELD

The disclosure generally relates to assessing hemodynamics using electrical impedance measurements, systems for assessing hemodynamics, and related methods.

### BACKGROUND

This section is intended to introduce the reader to various aspects of the art that may be related to aspects of the embodiments that are described or claimed below. The discussion is provided as background information to facilitate the understanding of the aspects of the embodiments of the present disclosure. As such, while the statements include useful background, these statements should not be construed as admissions of prior art.

Electrical Impedance Tomography (EIT) is a non-invasive imaging method that may be used to generate images or physiological measurements associated with a section of a subject (e.g., a patient or object) by collecting data using electrodes disposed along the perimeter of the subject. Potential applications of EIT include medical applications, in which real time tomographic images or physiological measurements of the human body may be useful. As an example, EIT may be used to perform non-invasive monitoring of cardio-respiratory systems, which may be particularly useful in patients under treatment in intensive care unit (ICU) environments.

In an EIT system, electrical signals (e.g., electric currents) may be injected in a perimeter of the subject being imaged (e.g., a patient torso). Electrical characteristics (e.g., voltages, electric potentials) resulting from the injected electrical signals may be collected at the perimeter of the subject. From the collected data, maps with an estimate of the electrical impedance in each portion (e.g., pixel, voxel) of the subject may be generated or reconstructed. This process may be repeated over time, and the sequence of tomography images may form a “video.” This video may be employed to provide physiological information to a physician or other clinical practitioner. Processing and/or quantification from the EIT data may also be performed and/or used to calculate physiological parameters and/or figures or merits, which may be used by a physician or other clinical practitioner.

### BRIEF SUMMARY

Embodiments of the present disclosure may include a method for performing hemodynamic assessment using an impedance-based device. The method may include determining a first impedance quantification associated with a first region of interest (ROI) in a first impedance image. The first impedance image may be associated with a first state of a patient prior to a maneuver. The method may further include determining a second impedance quantification associated with the first region of interest in a second impedance image. The second impedance image may be associated with a second state of the patient following the maneuver. The method may also include receiving at least one parameter associated with the maneuver. The method may further include determining an intrathoracic pressure change or a simulated replacement volume or both responsive to the at least one parameter associated with the maneuver. The method may also include determining at least one hemodynamic parameter in consideration of a difference between the first impedance quantification and the second impedance quantification, and at least one of the intrathoracic pressure change or the simulated replacement volume.

Other embodiments of the present disclosure may include a non-transitory computer-readable media storing instructions thereon. When executed by a processor the instructions may cause the processor to receive a sequence of impedance datasets. Each impedance dataset of the sequence of impedance datasets may be associated with a respective time instant. The instructions may further cause the processor to determine a pre-maneuver hemodynamic measurement from a first impedance dataset associated with a time instant prior to a maneuver. The instructions may also cause the processor to determine a post-maneuver hemodynamic measurement from a second impedance dataset associated with a second time instant following the maneuver. The instructions may further cause the processor to receive at least one parameter associated with the maneuver. The instruction may also cause the processor to determine a hemodynamic figure of merit using the pre-maneuver hemodynamic measurement, the post-maneuver hemodynamic measurement, and the at least one parameter associated with the maneuver.

Other embodiments of the present disclosure may include a system. The system may include an electrical impedance tomography (EIT) system. The EIT system may include a plurality of electrodes; and a controller coupled to the plurality of electrodes. The controller may include an interface module configured to collect EIT data from the plurality of electrodes. The controller may further include at least one processor; and at least one non-transitory computer-readable storage medium storing instructions thereon. When executed by the at least one processor, the instructions may cause the controller to receive a sequence of impedance datasets. The instructions may further cause the controller to generate, from each impedance dataset, a corresponding impedance image. The instructions may also cause the controller to determine a pre-maneuver hemodynamic measurement from a first region of interest (ROI) from at least one impedance image prior to a maneuver. The instructions may further cause the controller to determine a post-maneuver hemodynamic measurement from the first ROI from at least one impedance image following the maneuver. The instructions may also cause the controller to receive at least one parameter associated with the maneuver. The instructions may further cause the controller to determine, a hemodynamic figure of merit based at least on the pre-maneuver hemodynamic measurement, the post-maneuver hemodynamic measurement, and the at least one parameter associated with the maneuver.

### BRIEF DESCRIPTION OF THE DRAWINGS

Aspects of the present disclosure may be better understood by following the detailed description along with referenced drawings, in which:

FIG. 1 is a schematic diagram of a method that may be used to assess hemodynamics using an electrical impedance data according to one or more embodiments of the present disclosure;

FIG. 2 is a schematic diagram of an electrical impedance tomography (EIT) system that may be used to assess hemodynamics according to one or more embodiments of the present disclosure;

FIG. 3 is a schematic diagram of a method to obtain electrical impedance data using an EIT system according to one or more embodiments of the present disclosure;

FIG. 4 is a flow chart of a method for quantifying or assessing hemodynamics using EIT reconstructed data according to one or more embodiments of the present disclosure;

FIG. 5 is a diagram of a user interface system that may be used to facilitate and/or inspect the calculation of hemodynamic data from EIT reconstruction data according to one or more embodiments of the present disclosure;

FIG. 6 is a chart that illustrates an example of the quantification of a hemodynamic figure of merit from electrical impedance data, which may be used to assess hemodynamics according to one or more embodiments of the present disclosure;

FIG. 7 is a flowchart of a method to quantify a hemodynamic figure of merit from hemodynamic data according to one or more embodiments of the present disclosure;

FIG. 8 is a chart that illustrates a user interface that may facilitate the determination of the hemodynamic response of a patient using the methods described herein according to one or more embodiments of the present disclosure;

FIG. 9 is a comparative chart that illustrates the performance of a method to determine hemodynamics from electrical impedance data according to one or more embodiments of the present disclosure; and

FIG. 10 is a flowchart of a method of quantifying a hemodynamic figure of merit from hemodynamic data according to one or more embodiments of the present disclosure.

### DETAILED DESCRIPTION

The illustrations presented herein are not actual views of any EIT system, or any component thereof, but are merely idealized representations, which are employed to describe embodiments of the present disclosure.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, any relational term, such as “first,” “second,” etc., is used for clarity and convenience in understanding the disclosure and accompanying drawings, and does not connote or depend on any specific preference or order, except where the context clearly indicates otherwise.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

The present disclosure relates to electronic impedance tomography (EIT) methods and systems and, more specifically, to EIT systems capable of providing non-invasive hemodynamic measurements in real time or quasi-real time from impedance measurements.

One or more embodiments of the present disclosure are described below. To provide a concise description of these embodiments, certain features of an actual implementation may be omitted. It should be appreciated that in an actual implementation of the embodiments, as in any engineering or design project, several implementation-specific decisions are made to achieve the developers' specific goals and, as a result, implementations may vary from one another. Moreover, while a development effort might be complex and time consuming, certain developments would, nevertheless, be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, references to “one embodiment” or “an embodiment” of the present disclosure are not intended to be interpreted as excluding the existence of additional embodiments that also incorporate the recited features. Furthermore, the phrase A “based on” B is intended to mean that A is at least partially based on B.

As discussed herein, references may be made to “electrical impedance data.” As described herein, electrical impedance data relates to data obtained from an electrical impedance tomography (EIT) system or an electrical impedance measurement device, or any other data that relates to electrical characteristics (e.g., impedance, resistivity, capacitance, inductance, permittivity, etc.) of the measured subject (e.g., a patient, an object). Electrical impedance data refers to raw (i.e., unprocessed) data, filtered data, and/or tomographically reconstructed data. Tomographical reconstructions from electrical impedance data may be specifically referred herein as “electrical impedance images” or “electrical impedance videos.”

Electrical impedance images may be further described as “absolute impedance images,” “differential impedance images,” and “volumetric images” of a subject. Absolute impedance images, as discussed herein, relate to images in which the representation (e.g., value) of each pixel or voxel is associated to the impedance of the corresponding portion of the subject being imaged. Differential impedance images, as discussed herein, relate to images in which the representation (e.g., value) of each pixel or voxel is associated to a change of the impedance of the corresponding portion of the subject relative to a baseline image. Volumetric images, as discussed herein, relates to images in which the representation (e.g., value) of each pixel or voxel is associated with a volume of a biological fluid (e.g., air, blood, water, tissue type). Moreover, it should be understood that the impedance images or videos, as referred herein, may be tomographic images that may be two-dimensional (2-D) or three-dimensional (3-D), as well as tomographic movies with a time dimension.

As discussed above, due to the nature of the measurements and the speed of data acquisition and processing, electrical impedance-based systems, such as EIT, can be used to generate high-frequency, real-time physiological data that may be of assistance to physician during certain diagnostic and/or clinical procedures. Moreover, since impedance-based systems are generally non-invasive, they can be used with a wider range of patient populations in a broad variety of conditions. The present disclosure relates to the EIT systems and methods of operation thereof that can calculate physiological parameters associated with cardiovascular activity for hemodynamics assessment of a patient.

Hemodynamic assessment may be used to identify patients that may benefit from certain types of therapies or interventions. For example, volume replacement (i.e., fluid replacement) is commonly used in patients undergoing cardiac surgery, to improve their surgery outcomes. However, only a portion of the patients are fluid responsive and benefits from an increase in systolic volume (SV) and/or cardiac output (CO) in response to the volume replacement. Patients that are not fluid responsive may not benefit from the volume replacement and may be susceptible to potential side effects of such interventions. Therefore, a hemodynamic assessment may be used to prevent ineffective or potentially harmful use of volume replacement in these patients. More generally, hemodynamic assessment can be a powerful diagnostic tool in several clinical applications.

Conventional approaches for hemodynamic assessment may employ invasive techniques. As an example of such technique, certain systems employ catheters applied to a peripheral artery to obtain an invasive measurement of the blood pressure signal. The invasive blood pressure signal can be monitored during some external maneuver, as detailed below, and the response of the blood pressure signal to the maneuver may be quantified. The use of invasive techniques and methods to measure cardiac parameters may present some complications to certain patients and, thus, limit its application. Therefore, as discussed above, the use of non-invasive impedance-based devices and systems, such as EIT systems, may increase the application of hemodynamic assessments.

With the foregoing in mind, the present disclosure relates to the use of impedance-based systems, such as EIT systems, to calculate physiological parameters related to hemodynamic assessments, as discussed above. In some embodiments, the impedance-based system may employ filtering and/or reconstruction processing of impedance data to quantify physiological parameters in a region of interest (ROI) of the image. The hemodynamic parameters discussed herein may include pulse impedance variation (PIV), systolic volume (SV), systolic volume variation (SVV), cardiac output (CO), associated indices, and/or other hemodynamic figures of merit, as detailed below. In some embodiments, the impedance-based system may be coupled to other medical equipment, such as mechanical ventilation systems and/or electrocardiogram monitors, to receive additional clinical data for improved hemodynamic assessment and/or to provide calculated data. In some embodiments, the impedance-based system may include user interfaces (UIs) that may guide a clinical practitioner during the hemodynamic assessment.

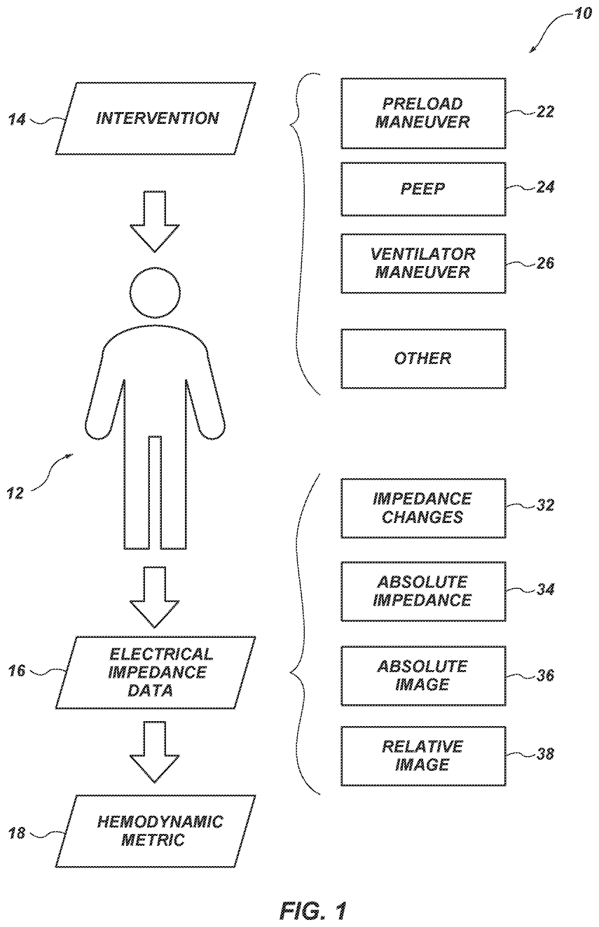

FIG. 1 illustrates a schematic diagram for a method 10 to perform hemodynamic assessment of a patient 12. In the hemodynamic assessment 10, the patient 12 may be subjected to an intervention 14 that may cause hemodynamic changes to the patient. The patient 12 may be monitored using an impedance-based device that collects electrical impedance data 16. The electrical impedance data 16 may include data from prior to the intervention 14, during the intervention 14, and/or after the intervention 14. The electrical impedance data 16 may be processed to obtain one or more hemodynamic-associated metrics 18. The hemodynamic metrics may be displayed to a practitioner or may be compared against threshold values to generate alarm signals or other types of display, as detailed below.

The intervention 14 may be any controlled intervention that can generate hemodynamic changes in the patient 12. In certain situations, a cardiac pre-load simulation maneuver 22 (e.g., a change in the patient's posture, the change in the position of limbs) may be used. In some embodiments, the impedance-based system may be configured to provide instructions to the practitioner using the pre-load simulation maneuver 22 during the hemodynamic assessment process. For example, the impedance-based system may provide instructions associated with the timing of the initiation and/or termination of the pre-load simulation maneuver 22 and/or specific instructions for the procedure (e.g., “raise the leg by 45 degrees”). The electrical impedance data 16 may be recorded in accordance with the provided instructions and may include metadata associated with the provided instructions.

In situations where the patient 12 is connected to a mechanical ventilator, ventilator maneuvers 26 may be used. As an example, a temporary change to the positive end-expiratory pressure (PEEP) parameter of the ventilator (PEEP maneuver 24) might be used to increase the intra-thoracic pressure in a controlled manner. In some embodiments, the impedance-based system may be configured to provide instructions to the practitioner to perform the ventilator maneuver 26 during the hemodynamic assessment process. For example, the impedance-based system may provide instructions associated with the timing of the initiation and/or termination of the ventilator maneuver 26 and/or specific instructions for the procedure (e.g., “increase the PEEP by 5 cmH2O” for a PEEP maneuver 24). In some embodiments, the impedance-based system may be connected to the mechanical ventilator to receive respiratory data from the ventilator, which may be included as metadata for the electrical impedance data 16. In some embodiments, the impedance-based system may include a flow and/or pressure sensor that may provide respiratory data associated with ventilation, which may be included as metadata for the electrical impedance data 16. In some embodiments, the impedance-based system may be configured to control the mechanical ventilator to perform the hemodynamic assessment. The electrical impedance data 16 may be recorded during the hemodynamic assessment in accordance with the ventilator maneuver 26 and may include respiratory data and/or ventilator controls as associated metadata.

The electrical impedance data 16 that can be used for hemodynamic assessment may include the relative impedances 32, absolute impedances 34, absolute tomographic images 36, relative tomographic images 38, and other such images. In some embodiments, the hemodynamic metric 18 may be separately calculated in different regions of interest (ROIs) of the subject. In those embodiments, the tomographic images 36, 38 may be used, as detailed below.

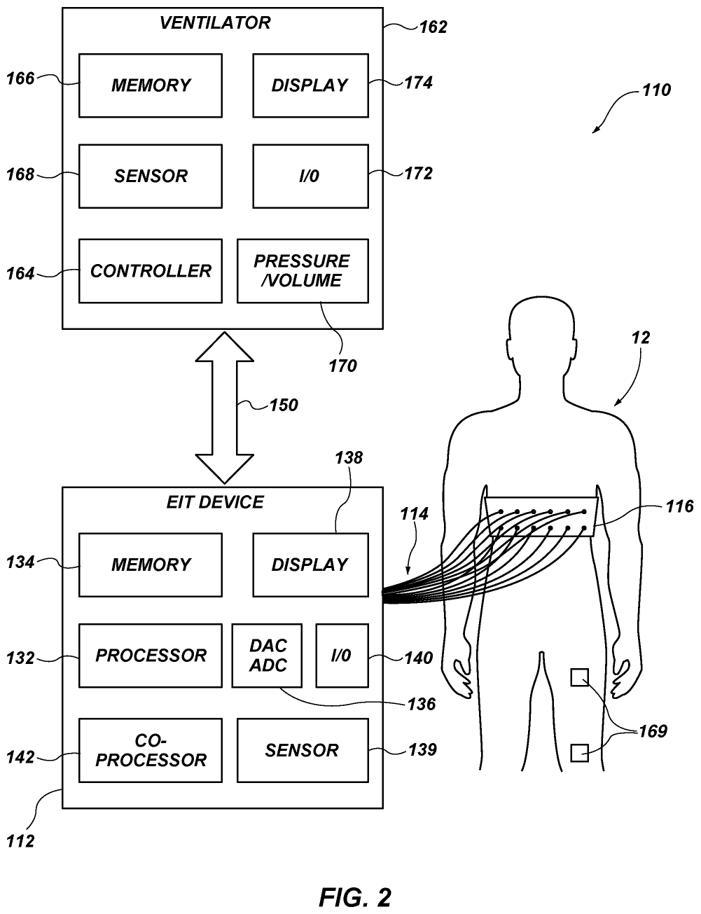

FIG. 2 provides a schematic diagram of a system 110 that includes an EIT device 112 used in conjunction with a mechanical ventilator 162 to perform hemodynamic assessment. It should be understood that, as discussed above, the hemodynamic assessment can be performed in the absence of mechanical ventilation such as that provided by the mechanical ventilator 162. The EIT device 112 that may be coupled through electrical leads 114 to an electrode belt 116 that may be placed on the patient 12. The electrode belt 116 may have one or more rows of electrodes, disposed along the perimeter of the electrode belt 116. The electrodes of the electrode belt 116 may be spatially distributed along the perimeter of the torso of patient 12. The EIT device 112 may inject electrical currents through electrodes of the electrode belt 116 and may collect the resulting electrical voltages. From the collected data, the EIT device 112 may calculate one or more hemodynamic metrics (e.g., hemodynamic metrics 18 of FIG. 1).

The EIT device 112 may perform filtering, reconstruction, and/or quantification algorithms, as detailed below. To that end, the computational device may include one or more processors 132 and one or more processing memory devices 134 (e.g., cache memory, random access memory (RAM)) to facilitate execution of the algorithms. The memory devices 134 may also include one or more protocols for performing the hemodynamic assessment. The protocols may include instructions to be provided to a practitioner and instructions to create or associate metadata to the impedance data. The memory devices 134 may also include one or more segmentation algorithms to automatically determine ROIs used in the hemodynamic assessment. In some embodiments, the segmentation algorithms may include principal component analysis (PCA).

In some embodiments, some or all of the algorithms described herein may be performed by a co-processor 142, which may be a second processor, a specialized processor such as a graphical processing unit (GPU), an application-specific integrated circuit (ASIC) designed to perform the algorithms, a field-programmable gate array (FPGA) containing configuration program that performs the algorithms, or any other dedicated electronic device.

The EIT device 112 may also include an interface module 136 to control the electrical currents injected into the electric leads 114 and measure the voltages between the electrical leads 114. The interface module 136 may include, among other things, analog signal generators, analog-to-digital converters, digital-to-analog converters, digital signal processors, filters, and impedance matching circuitry, to improve signal-to-noise ratio and decrease crosstalk.

In some embodiments, the EIT device 112 may include a display 138, which may be used to provide reconstructed images, hemodynamic charts, and/or hemodynamic indices, as well as diagnostic parameters that may be calculated from the EIT images. The display 138 may also be employed to provide instructions to a practitioner performing the hemodynamic assessment, as discussed above. In some embodiments, the display 138 may include or may be connected to a speaker or similar sound producing device to provide auditory alerts or auditory commands associated with the hemodynamic assessment. In some embodiments, the EIT device 112 may include sensors 139, which may include one or more of a pressure sensor, flow sensor, and flow sensor, a carbon dioxide sensor and/or any combination thereof. The sensors 139 may be configured to measure data, such as respiratory data from the EIT device 112 and/or the associated ventilator 162. Respiratory data obtained from sensors 139 may be used to facilitate the hemodynamic assessment process, as discussed above.

In some embodiments, the EIT device 112 may include input/output interfaces 140 such as network interfaces, hard disk interfaces, and/or peripheral interfaces to send or receive data that may facilitate the operations of EIT, as discussed herein. For example, the EIT device 112 may be connected to the ventilator device 162 over an interface 150 that is coupled to the input/output interface 140. The interface 150 may be used to carry control commands and/or data that may be used to facilitate the hemodynamic assessment process, as discussed above.

Moreover, the input/output interfaces 140 may also be connected to position sensors 169 that may be in contact with the patient or placed in an automated bed where the patient is disposed. For example, if a pre-load cardiac simulation maneuver (e.g., pre-load cardiac simulation maneuver 22) is employed by repositioning the patient or patient limb, the position sensors 169 may measure the actual position and provide to the EIT device 112 through the input/output interface. In some embodiments, the position sensor 169 may be a video recording device that records the patient accompanied by an image processing system capable of quantifying the relative position of the limbs from the recorded video. The measurements may be incorporated as metadata for the electrical impedance data.

In certain configurations of the system 110, such as the one illustrated in FIG. 2, the patient 12 may be connected to the mechanical ventilator 162. The ventilator 162 may include a controller 164 (e.g., a processor, a microcontroller) that may, in conjunction with one or more memory devices 166, control the operations of the ventilator 162. In some embodiments, the memory devices 166 may include protocols to perform ventilator maneuvers (e.g., ventilator maneuvers 26), to facilitate the hemodynamic assessment. The mechanical ventilator device 162 may include sensors 168, which may be a pressure sensor, flow sensor, and flow sensor, a carbon dioxide sensor. The ventilator device 162 may also include pumps 17, which may be pressure-controlled or volume-controlled pumps that provide respiratory support to the patient 12. The sensors 168 and pumps 170 may be controlled and/or monitored by the controller 164.

In some embodiments, the ventilator device 162 may include input/output interfaces 172 such as network interfaces, hard disk interfaces, and/or peripheral interfaces to send or receive data that may facilitate the respiratory support operations. The input/output interfaces 172 may be used to connect the ventilator 162 to the EIT device 112 over an interface 150, as discussed above. The interface 150 may be used to carry control commands or instructions from the EIT device 112 and to provide respiratory data from sensors 168 to the EIT device 112 to facilitate the hemodynamic assessment process, as discussed above.

In some embodiments, the ventilator device 162 may include a display 174, which may be used to provide respiratory charts, indices, and other physiological parameters associated with the respiratory support provided to the patient. In certain embodiments, the ventilator device 162 may be configured to facilitate hemodynamic assessments. In such devices, the display 174 may be configured to provide instructions to a practitioner performing the hemodynamic assessment, as discussed above. In some embodiments, the display 174 may include or may be connected to a speaker or similar sound producing device to provide auditory alerts or auditory commands associated with the hemodynamic assessment.

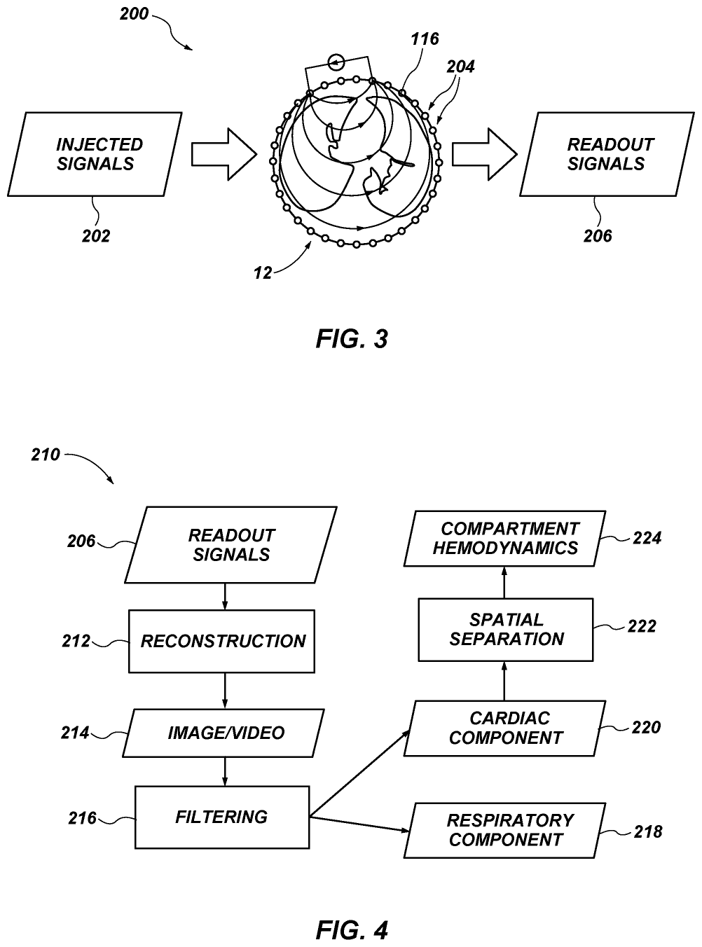

FIG. 3 illustrates a schematic diagram 200 for the performance of impedance measurements. Electrical signals 202 may be injected into pairs of electrodes 204 disposed along the electrode belt 116 around the patient 12. The electric signals 202 may be injected in a pre-programmed order, and the resulting electrical potentials 206 between pairs of electrodes 204 may be read out. The collection of electrical potentials 206 may form the raw impedance data that may be used for hemodynamic assessment discussed herein.

FIG. 4 illustrates a flow chart for a method 210 of calculation of hemodynamics parameters using EIT systems, such as the system 110 of FIG. 3. The collected electrical potentials 206 may be used to perform tomographic reconstruction (box 212) to obtain a series of impedance images 214. The tomographic reconstruction (box 212) may employ an algorithm that provides absolute or relative impedance images. The reconstruction algorithm of box 212 may include filters or other signal processing tools to mitigate noise and other artifacts. The reconstruction algorithm may be model-based algorithms.

The series of impedance images 214 may be further filtered (box 216) to separate individual component contributions. For example, a respiratory component 218 and/or a cardiac component 220 may be extracted from the impedance images 214. Since the respiratory frequency is usually lower than the cardiac frequency, the filtering algorithm of box 216 may employ frequency-based filtering over the time dimension of an impedance images 214. Examples of frequency-based filtering include high-pass filtering, low-pass filtering, band-pass filtering, Fourier-based filtering, moving average filters, autoregressive filters, autoregressive moving average (ARMA) filters, wavelet filters, state-space filters, or any other filter capable of separating components based on their frequency. Moreover, since perturbations from cardiac events and respiratory events are concentrated in different regions, the filtering algorithm of box 216 may employ ROIs to facilitate the extraction of the cardiac component 220. It should be understood that box 216 may employ any filtering algorithm capable of providing a quantifiable cardiac component 220, or separating it from the respiratory component 218, which may be dominant.

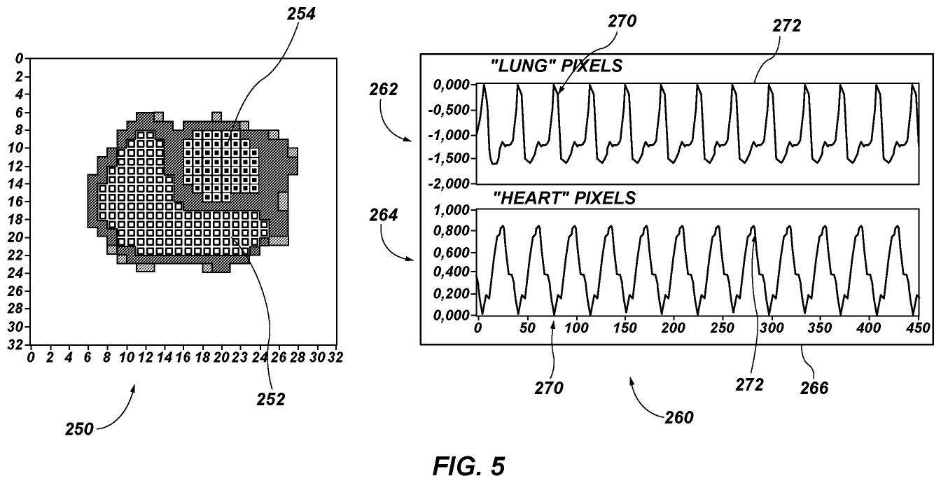

The cardiac component 220 of the impedance images 214 may be quantified in separated ROI regions (cardiac component 222). For example, pixels associated with the cardiac tissue and pixels associated with lung tissue may be placed in separate ROIs. In some embodiments, this process may be implemented using principal component analysis (PCA) methods. This spatial separation may improve the quality of the impedance data for hemodynamic assessment by reducing crosstalk between tissues that respond incoherently to cardiac events, as detailed below in the discussion of FIG. 5. The quantification may provide one or more physiological parameters 224 that may be used to perform the hemodynamic assessment.

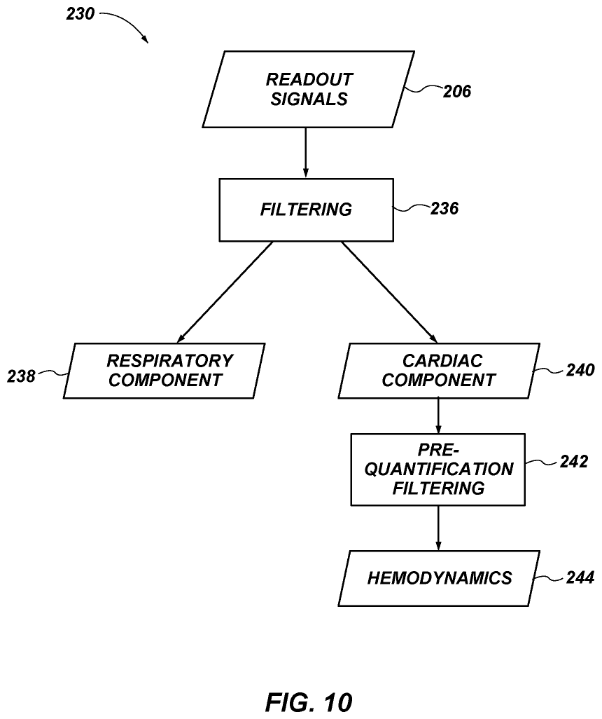

FIG. 10 illustrates a flow chart for a method 230 of calculation of hemodynamics for electrical impedances systems, such as the EIT system 110 of FIG. 3. Method 230 can be used to calculate hemodynamics parameters without necessarily performing an image reconstruction. In method 230 the electrical potentials 206, which may form or be arranged as a data matrix (e.g., a time series of matrices, wherein each element of the matrix is an electrical potential associated with a pair of electrodes), may be used directly. In this method filtering (box 236) may include filters or other signal processing tools to mitigate noise or other artifacts. Moreover, the filtering in box 236 may be used to separate individual component contributions. For example, a respiratory component 238 and/or a cardiac component 240 may be extracted from the electrical potentials 206. Since the respiratory frequency is usually lower than the cardiac frequency, the filtering algorithm of box 236 may employ frequency-based filtering over the time dimension over the electrical potentials 206. Examples of frequency-based filtering include high-pass filtering, low-pass filtering, band-pass filtering, Fourier-based filtering, moving average filters, autoregressive filters, autoregressive moving average (ARMA) filters, wavelet filters, state-space filters, or any other filter capable of separating components based on their frequency. It should be understood that box 236 may employ any filtering algorithm capable of providing a quantifiable cardiac component 240, or separating it from the respiratory component 238, which may be dominant.

The cardiac component 240 of the electrical potentials 206 may be further filtered (box 242) to improve the performance of hemodynamic quantification. For example, certain elements (e.g., signals from specific pairs of electrodes) may be more sensitive to cardiac variations whereas other elements may be more sensitive to respiratory variations. In some embodiments, this process may be implemented using principal component analysis (PCA) methods. This further filtering may improve the quality of the hemodynamic assessment by reducing crosstalk between data elements that respond incoherently to cardiac events. The quantification may provide one or more physiological parameters 244 that may be used to perform the hemodynamic assessment.

In some embodiments, quantification of the cardiac component 220 may be facilitated by the heartbeat markers, which may provide synchronization signals associated with an event of the cardiac cycle. For example, the heartbeat marker may be associated with an atrial systolic peak or a diastolic peak, an aortic pressure peak, or any of the P, Q, R, S, or T peaks of an electrocardiograph signal (ECG). The heartbeat marker may be extracted from a cardiac electrical signal, which may be acquired with a separate patient monitor. In some embodiments, the heartbeat markers may be obtained using the electrodes of the EIT device (e.g., electrodes in electrode belt 116 of EIT device 112).

As discussed above, the impedance data may be accompanied by metadata obtained by, for example, positional sensors, video images, mechanical ventilators, practitioner inputs, and/or flow/pressure sensors. The metadata might facilitate the quantification of the physiological parameters 224. The metadata may provide, for example, time information (e.g., time of initiation of the maneuver, time of termination of the maneuver, duration of the maneuver), maneuver-specific information (e.g., type of maneuver, volume simulated, change in volume, tidal volume), ventilator-specific information (e.g., frequency of breathing, respiratory flow, respiratory pressure, time of inspiration, time of expiration, spontaneous expiration event, tidal volume), and/or other physiological information (e.g., pulse, oxygen concentration, heartbeat markers). The metadata may be used to perform, for example, cardiac component extraction (e.g., heartbeat synchronization), automatic segmentation of ROIs, filtering and/or extraction of the cardiac component 222, and/or calculations of the hemodynamic figures of merit.

FIG. 5 illustrates user interface elements of an EIT device (e.g., EIT device 112) that might be provided in a display (e.g., display 138). Specifically, FIG. 5 illustrates an ROI map 250. The illustrated ROI map 250 includes a lung ROI 252, which includes pixels where lung tissue is the dominant tissue. The illustrated ROI map 250 also includes a heart ROI 254, which includes pixels where cardiac tissue is dominant. FIG. 5 also includes impedance plethysmograph charts 260. Plethysmograph charts 260 includes a lung plethysmograph 262, which displays impedance variations in the lung ROI 252 and a heart plethysmograph 264, which displays impedance variations in the heart ROI 254. The plethysmograph charts 260 shows the changes in impedance along a time axis 266.

The illustrated plethysmograph charts 260 displays the cardiac component (e.g., cardiac component 220 of FIG. 4) and the respiratory component (e.g., respiratory component 218 of FIG. 4) was filtered out. That is, lung plethysmograph 262 illustrates the impedance variations in the lung ROI 252 as a result of cardiovascular events and heart plethysmograph 264 illustrates the impedance variations in the heart ROI 254 as a result of the same cardiovascular event. For example, at time 270 the heart plethysmograph 264 present a valley, which represents an increase in tissue fluid that causes a drop in the impedance. At the same time 270, the lung plethysmograph 262 presents a peak, which represents an increase in air associated with a drop in the lung perfusion. This behavior can be associated with cardiac diastole. By contrast, at time 272, the lung plethysmograph 262 presents a value, which may be associated with lung perfusion, and the heart plethysmograph presents a peak, which may be associated with a decrease in tissue fluid. This behavior can be associated with systole. As such, the use of different ROIs may allow an improved ability for the EIT device 112 to discriminate between different cardiovascular events, which can improve the calculation of figures of merit for hemodynamic events.

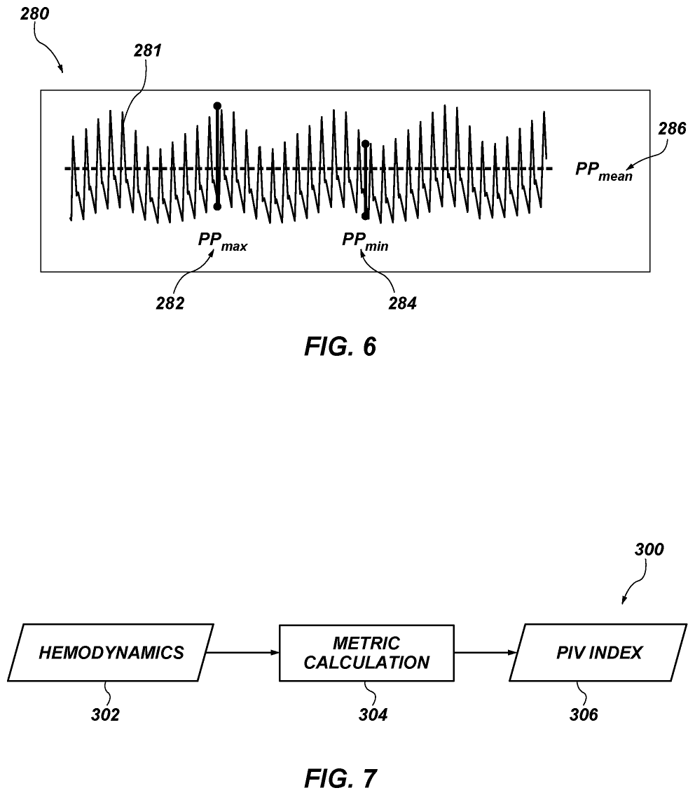

FIG. 6 provides an illustration of a hemodynamic figure of merit, the pulse impedance variation (PIV) that may be calculated using the systems and methods discussed herein. The chart 280 illustrates an impedance plethysmograph 281 of the cardiac component, which may be obtained from pixels in a lung ROI (e.g., lung ROI 252) over time. It should be understood that the cardiac component may be obtained from pixels in either the lung ROI 252 or the heart ROI 254, as well as from non-segmented pixels. The impedance plethysmograph 281 illustrates the variation of the cardiac component (e.g., cardiac component 222) across multiple breathing cycles (e.g., respiratory component 218). During expiration, represented at time 282, the intra-thoracic pressure is lower and, therefore, the amplitude PP_{MAX} of the plethysmograph 281 may be at its peak. During inspiration, represented at time 284, the amplitude PPMIN of the plethysmograph 281 may be the lowest. The chart also illustrates the mean pressure PPMEAN 286. From these measurements, extracted from the EIT system plethysmograph, a pulse impedance variation (PIV) index may be automatically calculated by the EIT system using the following expression:

$$PIV = \frac{P {P}_{MAX} - P {P}_{MIN}}{P {P}_{MEAN}}$$

The PIV index indicates the patient's fluid responsiveness. In some embodiments, the EIT system may store standardized threshold values for the PIV and may automatically provide an indication that the PIV index of a patient is above or below a standardized threshold value.

FIG. 7 provides a flowchart for the method 300 to calculate the PIV, discussed above. From a hemodynamic data 302 (e.g., plethysmograph of a cardiac ROI), the EIT system may calculate one or more metrics (box 304). The EIT system may employ the above-described metadata to facilitate calculation of the metrics. For example, respiratory data may be used to determine the moment of inspiration or expiration to collect the PP_{MIN} or PP_{MAX}, respectively. From the one or more metrics, the EIT system may, in box 304, derive an index 306, such as the PIV index. The index 306 may be associated with the fluid responsiveness of the patient. While FIG. 7 illustrates an amplitude-based figure of merit, it should be understood that other signal characteristics, such as baseline, phase lag, or the delay between a heartbeat (e.g., a heartbeat marker discussed above) and an oscillation (i.e., propagation time) may be used.

FIG. 8 illustrates a user interface 320 that may be used to provide hemodynamic information to the practitioner. User interface 320 may include a plethysmograph chart 322 and a stroke volume chart 324. The data for the plethysmograph chart 322 and the stroke volume chart 324 may be calculated from impedance data calculated from the EIT system. The stroke volume chart 324 may be calculated from the cardiac component measured in the heart or lung ROIs. The stroke volume chart 324 may be a chart of the absolute stroke volume, or a chart of percentual changes relative to a reference stroke volume. The user interface also includes physiological parameter display 326 showing the current stroke volume, parameter display 328 showing the heart rate, and parameter display 330 showing the current cardiac output. These parameter displays may provide quantitative information to assist the practitioner.

FIG. 8 also illustrates the performance of the EIT system during maneuvers. In the period between time events 332 and 334, the PEEP was changed from 8 cmH2O to 13 cmH2O and returned to 8 cmH2O. At time 336, the leg of the patient was raised and lowered. The stroke volume chart 324 shows that the patient appears to present fluid responsiveness. At time 338, the patient underwent a volume replacement procedure and presented an increase in systolic volume, as expected from a patient that is fluid responsive.

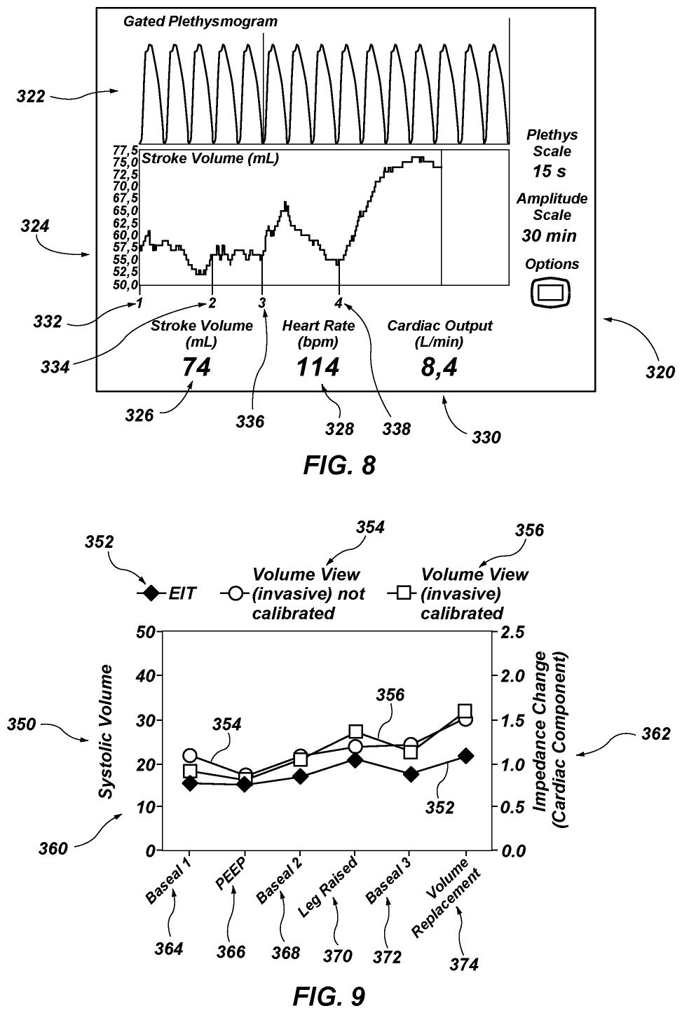

FIG. 9 is a chart 350 that compares an implementation of one of the methods presented herein with an invasive method to assess patient hemodynamics. The chart 350 presents impedance changes in the cardiac ROI 362 estimated using an EIT system (EIT dataset 352) and compares it to the systolic volume 360 obtained using a VolumeView (Edwards, Inc.) device. The VolumeView data collected was uncalibrated (VolumeView uncalibrated dataset 354) and calibrated (VolumeView calibrated dataset 356). The chart 350 also shows, initially, a basal level 364. Following the basal level 364, a PEEP maneuver 366 is initiated. Following the PEEP maneuver 366, the patient is returned to the basal level 368. A cardiac pre-load simulation maneuver 370, performed by raising the patient's leg, is also demonstrated. Following maneuver 370, the patient is returned to the basal level 372. Finally, the patient receives volume replacement therapy 374.

Non-limiting example embodiments of the present disclosure may include:

Embodiment 1: A method for performing hemodynamic assessment using an impedance-based device, comprising: determining a first impedance quantification associated with a first region of interest (ROI) in a first impedance image, wherein the first impedance image is associated with a first state of a patient prior to a maneuver; determining a second impedance quantification associated with the first region of interest in a second impedance image, wherein the second impedance image is associated with a second state of the patient following the maneuver; receiving at least one parameter associated with the maneuver; determining an intrathoracic pressure change or a simulated replacement volume or both responsive to the at least one parameter associated with the maneuver; and determining at least one hemodynamic parameter in consideration of a difference between the first impedance quantification and the second impedance quantification, and at least one of the intrathoracic pressure change or the simulated replacement volume.

Embodiment 2: The method of embodiment 1, wherein the at least one parameter associated with the maneuver comprises one or more of a positive end-expiratory pressure (PEEP) value, a PEEP change, an inspiration time, an expiration time, a respiratory cycle period, a tidal volume value, a tidal volume change, or a position change.

Embodiment 3: The method of embodiment 2, wherein the at least one parameter associated with the maneuver is received from one or more of a mechanical ventilation device, a pressure sensor configured to measure a respiratory system of the patient, a flow sensor configured to measure the respiratory system of the patient, or a carbon dioxide sensor configured to measure the respiratory system of the patient.

Embodiment 4: The method of any one of embodiments 1-3, wherein the at least one parameter associated with the maneuver is received via an input device of the impedance-based device from an external device, from a sensor disposed within the impedance-based device, or from both.

Embodiment 5: The method of any one of embodiments 1-4, wherein the method further comprises: determining a respiratory component data from impedance data generated by the impedance-based device; and generating the at least one parameter associated with the maneuver from the respiratory component data.

Embodiment 6: The method of any one of embodiments 1-5, wherein the first ROI is associated with a cardiac region or a lung region, or both.

Embodiment 7: The method of any one of embodiments 1-6, wherein the maneuver comprises a cardiac pre-load simulation maneuver comprising one or more of a change in a posture of the patient or a change in a position of at least one limb.

Embodiment 8: The method of any one of embodiments 1-7, wherein the maneuver comprises a mechanical ventilation maneuver.

Embodiment 9: The method of any one of embodiments 1-8, further comprising: acquiring a first plurality of electrical impedance signals; reconstructing the first impedance image from the first plurality of electrical impedance signals; acquiring a second plurality of electrical impedance signals; and reconstructing the second impedance image from the second plurality of electrical impedance signals.

Embodiment 10: The method of any one of embodiments 1-9, wherein the at least one hemodynamic parameter comprises one or more of a pulse impedance variation (PIV), a cardiac output (CO), a systolic volume (SV), a change in PIV, a change in CO, or a change of SV.

Embodiment 11: A non-transitory computer-readable media storing instructions thereon that, when executed by a processor causes the processor to perform steps comprising: receive a sequence of impedance datasets, wherein each impedance dataset of the sequence of impedance datasets is associated with a respective time instant; determine a pre-maneuver hemodynamic measurement from a first impedance dataset associated with a time instant prior to a maneuver; determine a post-maneuver hemodynamic measurement from a second impedance dataset associated with a second time instant following the maneuver; receiving at least one parameter associated with the maneuver; and determining a hemodynamic figure of merit using the pre-maneuver hemodynamic measurement, the post-maneuver hemodynamic measurement, and the at least one parameter associated with the maneuver.

Embodiment 12: The non-transitory computer-readable media of embodiment 11, wherein the non-transitory computer-readable media is configured to be executed by an electrical impedance tomography (EIT) device processor or by a general-purpose computer processor coupled to an EIT device, and wherein the instructions cause the EIT device process to: reconstruct a first impedance image from the first impedance dataset; determine the pre-maneuver hemodynamic measurement from a first region of interest (ROI) in the first impedance image; and reconstruct a second impedance image from the second impedance dataset prior to the determination of the post-maneuver hemodynamic measurement; and determine the post-maneuver impedance image from the first ROI in the second impedance image.

Embodiment 13: The non-transitory computer-readable media of any one of embodiments 11 or 12, wherein the maneuver comprises a mechanical ventilator maneuver and wherein the instructions cause the processor to request a mechanical ventilator to automatically perform the mechanical ventilator maneuver prior to the determination of the post-maneuver hemodynamic measurement and following the determination of the pre-maneuver hemodynamic measurement.

Embodiment 14: The non-transitory computer-readable media of any one of embodiments 11-13, wherein instructions cause the processor to determine an intrathoracic pressure change or a simulated replacement volume based at least in part on the at least one parameter associated with the maneuver.

Embodiment 15: The non-transitory computer-readable media of any one of embodiments 11-14, wherein the at least one parameter associated with the maneuver is received from a mechanical ventilator, a flow sensor, a pressure sensor, a volume sensor, an electrical impedance measurement device, or an electrical impedance tomography (EIT) device.

Embodiment 16: The non-transitory computer-readable media of any one of embodiments 11-15, wherein the pre-maneuver hemodynamic measurement and the post-maneuver hemodynamic measurement comprises a pulse impedance variation (NV), a cardiac output (CO), or a systolic volume (SV), or any combination thereof.

Embodiment 17: The non-transitory computer-readable media of any one of embodiments 11-16, wherein the hemodynamic figure of merit comprises a difference between the pre-maneuver hemodynamic measurement and the post-maneuver hemodynamic measurement.

Embodiment 18: The non-transitory computer-readable media of embodiment 17, wherein computer-readable media comprises instruction that cause the processor to compare the hemodynamic figure of merit with a threshold level to determine fluid responsiveness.

Embodiment 19: A system comprising: an electrical impedance tomography (EIT) system, comprising: a plurality of electrodes; and a controller coupled to the plurality of electrodes, the controller comprising: an interface module configured to collect EIT data from the plurality of electrodes; at least one processor; and at least one non-transitory computer-readable storage medium storing instructions thereon that, when executed by the at least one processor, cause the controller to: receive a sequence of impedance datasets; generate, from each impedance dataset, a corresponding impedance image; determine a pre-maneuver hemodynamic measurement from a first region of interest (ROI) from at least one impedance image prior to a maneuver; determine a post-maneuver hemodynamic measurement from the first ROI from at least one impedance image following the maneuver; receive at least one parameter associated with the maneuver; and determine, based at least on the pre-maneuver hemodynamic measurement, the post-maneuver hemodynamic measurement, and the at least one parameter associated with the maneuver, a hemodynamic figure of merit.

Embodiment 20: The system of embodiment 19, comprising a mechanical ventilator that comprises an input/output interface coupled to an input/output interface of the EIT system, wherein the mechanical ventilator comprises a computational device configured to transmit the at least one parameter associated with the maneuver to the EIT system.

Embodiment 21: The system of embodiment 20, wherein the at least one parameter associated with the maneuver comprises a positive end-expiratory pressure (PEEP), a PEEP change, an inspiration time, an expiration time, a respiratory cycle period, a tidal volume or any combination thereof.

Embodiment 22: The system of any one of embodiments 20 or 21, wherein the instructions cause the controller to transmit at least one control command to the mechanical ventilator associated with a maneuver.

Embodiment 23: The system of any one of embodiments 19-22, further comprising an automated bed comprising an input/output interface coupled to an input/output interface of the EIT system, wherein the automated bed is configured into a plurality of bed positions and wherein the automated bed is configured to transmit bed position data to the EIT system, and wherein receiving the at least one parameter associated with the maneuver comprises receiving the bed position data.

Embodiment 24: The system of any one of embodiments 19-23, comprising a position sensor configured to measure position data of a patient connected to the EIT system, and wherein receiving the at least one parameter associated with the maneuver comprises receiving the position data from the position sensor.

Embodiment 25: The system of embodiment 24, wherein the position sensor comprises a video sensor or a contact sensor.

Embodiment 26: The system of any one of embodiments 19-25, wherein the EIT system comprises a respiratory sensor configured to provide the at least one parameter associated with the maneuver.

Embodiment 27: The system of any one of embodiments 19-26, comprising a respiratory sensing device configured to provide the at least one parameter associated with the maneuver. The embodiments described in the present disclosure may be susceptible to various modifications and alternative forms, and specific embodiments have been shown by way of example in the drawings and have been described in detail herein. It should, however, be understood that the disclosure is not intended to be limited to the particular embodiments disclosed and that the disclosure covers all modifications, combinations, equivalents, and alternatives falling within the spirit and scope of the embodiments as defined by the following appended claims and legal equivalents thereof . In addition, the techniques and systems claimed herein refer to practical and useful embodiments and may be applied to concrete examples of a practical nature that demonstrably improve the present technical field and, as such, are not abstract, intangible or purely theoretical. Moreover, in claims that are appended to the end of this specification and contain one or more elements designated as “means for [performing a function] . . . ” or “step for [performing a function] . . . ,” it is intended that such elements are to be interpreted under 35 U.S.C. 112(f). For any claims containing elements designated in any other manner, however, it is intended that such elements are not to be interpreted under 35 U.S.C. 112(f).

1. A method for performing hemodynamic assessment using an impedance-based device, prior to an introduction of a replacement volume comprising:
determining a first impedance quantification associated with a first region of interest (ROI) in a first impedance image, wherein the first impedance image is associated with a first state of a patient prior to an external maneuver, wherein the external maneuver causes an intrathoracic pressure change, a simulated replacement volume, or both;
performing the external maneuver;
determining a second impedance quantification associated with the first region of interest in a second impedance image, wherein the second impedance image is associated with a second state of the patient following the external maneuver;
receiving at least one parameter associated with the external maneuver;
determining the intrathoracic pressure change or the simulated replacement volume or both responsive to a change to the at least one parameter associated with the external maneuver; and
determining at least one hemodynamic parameter in consideration of a difference between the first impedance quantification and the second impedance quantification.

2. The method of claim 1, wherein the at least one parameter associated with the maneuver comprises one or more of a positive end-expiratory pressure (PEEP) value, a PEEP change, an inspiration time, an expiration time, a respiratory cycle period, a tidal volume value, a tidal volume change, or a position change.

3. The method of claim 2, wherein the at least one parameter associated with the maneuver is received from one or more of a mechanical ventilation device, a pressure sensor configured to measure a respiratory system of the patient, a flow sensor configured to measure the respiratory system of the patient, or a carbon dioxide sensor configured to measure the respiratory system of the patient.

4. The method of claim 1, wherein the at least one parameter associated with the maneuver is received via an input device of the impedance-based device from an external device, from a sensor disposed within the impedance-based device, or from both.

5. The method of claim 1, wherein the method further comprises:
determining a respiratory component data from impedance data generated by the impedance-based device; and
generating the at least one parameter associated with the maneuver from the respiratory component data.

6. The method of claim 1, wherein the first ROI is associated with a cardiac region or a lung region, or both.

7. The method of claim 1, wherein the maneuver comprises a cardiac pre-load simulation maneuver comprising one or more of a change in a posture of the patient or a change in a position of at least one limb.

8. The method of claim 1, wherein the maneuver comprises a mechanical ventilation maneuver.

9. The method of claim 1, further comprising:
acquiring a first plurality of electrical impedance signals;
reconstructing the first impedance image from the first plurality of electrical impedance signals;
acquiring a second plurality of electrical impedance signals; and
reconstructing the second impedance image from the second plurality of electrical impedance signals.

10. (canceled)

11. A non-transitory computer-readable media storing instructions thereon that, when executed by a processor causes the processor to perform steps comprising:
receive a sequence of impedance datasets, wherein each impedance dataset of the sequence of impedance datasets is associated with a respective time instant prior to an introduction of a replacement volume;
determine a pre-maneuver hemodynamic measurement from a first impedance dataset associated with a time instant prior to an external maneuver, wherein the external maneuver causes an intrathoracic pressure change, a simulated replacement volume, or both;
determine a post-maneuver hemodynamic measurement from a second impedance dataset associated with a second time instant following the external maneuver;
receive at least one parameter associated with the external maneuver; and
determine a hemodynamic figure using the pre-maneuver hemodynamic measurement, and the post-maneuver hemodynamic measurement.

12. The non-transitory computer-readable media of claim 11, wherein the non-transitory computer-readable media is configured to be executed by an electrical impedance tomography (EIT) device processor or by a general-purpose computer processor coupled to an EIT device, and wherein the instructions cause the EIT device process to:
reconstruct a first impedance image from the first impedance dataset;
determine the pre-maneuver hemodynamic measurement from a first region of interest (ROI) in the first impedance image; and
reconstruct a second impedance image from the second impedance dataset prior to the determination of the post-maneuver hemodynamic measurement; and
determine the post-maneuver impedance image from the first ROI in the second impedance image.

13. The non-transitory computer-readable media of claim 11, wherein the maneuver comprises a mechanical ventilator maneuver and wherein the instructions cause the processor to request a mechanical ventilator to automatically perform the mechanical ventilator maneuver prior to the determination of the post-maneuver hemodynamic measurement and following the determination of the pre-maneuver hemodynamic measurement.

14. The non-transitory computer-readable media of claim 11, wherein instructions cause the processor to determine an intrathoracic pressure change or a simulated replacement volume based at least in part on a change in the at least one parameter associated with the maneuver.

15. The non-transitory computer-readable media of claim 11, wherein the at least one parameter associated with the maneuver is received from a mechanical ventilator, a flow sensor, a pressure sensor, a volume sensor, an electrical impedance measurement device, or an electrical impedance tomography (EIT) device.

16-18. (canceled)

19. A system comprising:
an electrical impedance tomography (EIT) system, comprising:
a plurality of electrodes; and
a controller coupled to the plurality of electrodes, the controller comprising:
an interface module configured to collect EIT data from the plurality of electrodes;
at least one processor; and
at least one non-transitory computer-readable storage medium storing instructions thereon that, when executed by the at least one processor, cause the controller to:
receive a sequence of impedance datasets prior to an introduction of a replacement volume;
generate, from each impedance dataset, a corresponding impedance image;
determine a pre-maneuver hemodynamic measurement from a first region of interest (ROI) from at least one impedance image prior to an external maneuver, wherein the external maneuver causes an intrathoracic pressure change, a simulated replacement volume, or both;
determine a post-maneuver hemodynamic measurement from the first ROI from at least one impedance image following the external maneuver;
receive at least one parameter associated with the external maneuver; and
determine a hemodynamic figure based at least on the pre-maneuver hemodynamic measurement, and the post-maneuver hemodynamic measurement.

20. The system of claim 19, comprising a mechanical ventilator that comprises an input/output interface coupled to an input/output interface of the EIT system, wherein the mechanical ventilator comprises a computational device configured to transmit the at least one parameter associated with the maneuver to the EIT system.

21. The system of claim 20, wherein the at least one parameter associated with the maneuver comprises a positive end-expiratory pressure (PEEP), a PEEP change, an inspiration time, an expiration time, a respiratory cycle period, a tidal volume or any combination thereof.

22. The system of claim 20, wherein the instructions cause the controller to transmit at least one control command to the mechanical ventilator associated with a maneuver.

23. The system of claim 19, further comprising an automated bed comprising an input/output interface coupled to an input/output interface of the EIT system, wherein the automated bed is configured into a plurality of bed positions and wherein the automated bed is configured to transmit bed position data to the EIT system, and wherein receiving the at least one parameter associated with the maneuver comprises receiving the bed position data.

24. The system of claim 19, comprising a position sensor configured to measure position data of a patient connected to the EIT system, and wherein receiving the at least one parameter associated with the maneuver comprises receiving the position data from the position sensor.

25-27. (canceled)
